(19) United States
(12) Patent Application Publication
(10) Pub. No.: US 2025/0302649 A1
(43) Pub. Date: Oct. 2, 2025

(54) STENT DEPLOYMENT APPARATUS FOR DEPLOYING A VASCULAR STENT AT A DEPLOYMENT SITE IN A BLOOD VESSEL OF VASCULATURE OF A BODY

(71) Applicants: Stan Batiste, Granite Bay, CA (US); Daniel Batiste, Granite Bay, CA (US)

(72) Inventors: Stan Batiste, Granite Bay, CA (US); Daniel Batiste, Granite Bay, CA (US)

(21) Appl. No.: 19/228,483

(22) Filed: Jun. 4, 2025

Related U.S. Application Data
(63) Continuation-in-part of application No. 19/088,244, filed on Mar. 24, 2025.
(60) Provisional application No. 63/569,427, filed on Mar. 25, 2024.
(60) Provisional application No. 63/651,885, filed on May 24, 2024.

(51) Int. Cl. A61F 2/966; A61F 2/954; A61F 2/958

(52) U.S. Cl. CPC A61F 2/966; A61F 2/954; A61F 2/958

(57) ABSTRACT

A stent deployment apparatus is configured to be contained within a deployment sheath, and deploy a vascular stent at a deployment site in a blood vessel of vasculature of a body. The stent deployment apparatus includes a delivery sheath, a pusher shaft coupled to the delivery sheath, and at least one stent assist bar associated with the delivery sheath and movable between a retracted position and a deployed position responsive to separation from the deployment sheath, the at least one stent assist bar being adapted to be positioned in relation to a known feature of the vasculature. The pusher shaft is movable with respect to delivery sheath in order to vary the spatial relationship between the vascular stent and the at least one stent assist bar, and thereby dispose the vascular stent to a desired spacing within the blood vessel.

### CROSS-REFERENCE TO RELATED APPLICATIONS

This application is a continuation-in-part patent application, which claims priority to and claims the benefit of U.S. patent application Ser. No. 19/088,244 filed on Mar. 24, 2025, which claims priority to and claims the benefit of U.S. Provisional Application No. 63/569,427 filed on Mar. 25, 2024, as well as U.S. Provisional Application No. 63/651,885 filed on May 24, 2024.

### FIELD OF THE INVENTION

This invention relates to vascular stents, and in particular to systems for precise deployment of vascular stents by minimally invasive procedures.

### BACKGROUND OF THE INVENTION

Vascular stents were developed in the 1980s and started to gain widespread acceptance in the 1990s following FDA approval for the repair and remediation of serious vascular conditions such as coronary heart disease, peripheral artery disease, and aneurysms. Stents are fabricated as tubular mesh structures made of metal, polymer or fabric, and may include a polymer coating designed for elution of anti-fibrotic drugs and retarding encapsulation. The stents can be either covered (commonly using PTFE) or uncovered, the latter generally referred to as “bare metal” stents. The mesh structure is collapsible to a small cylindrical structure for intravenous catheter delivery to the site of the affliction, and can thereafter be expanded to exert radial pressure in a vessel, either automatically by spring-like construction or by inflation of a balloon catheter inside the lumen of the stent. The radial pressure of an expanded stent maintains the stent in its implant location where it will compress plaque obstruction of the vessel or support and strengthen the wall of the vessel at the location of a developing aneurysm.

An attractive feature of vascular stents is that they can be implanted by a minimally invasive surgical procedure using a catheter that is threaded through the vascular system to the site requiring repair. The minimal bodily stress imposed by such procedures enables most patients to recover quickly and resume normal lifestyles. Catheter delivery is made possible by the use of real-time x-ray imaging (fluoroscopy), enabling the clinician to guide the catheter to the site in the vasculature where the stent must be delivered and implanted. This procedure, however, requires a great deal of skill and expertise, as the image guidance provided by fluoroscopy is minimal. Since fluoroscopy is an x-ray modality, it cannot provide distinct images of soft tissue structures like blood vessels. Consequently the clinician is mainly observing bones and the catheterization apparatus, the latter usually bearing radiopaque markings, and is relying largely on prior experience and educated estimation while trying to deploy the stent at the exact location in the vasculature where it is needed. Further complicating the procedure is the fact that the insertion and manipulation of the catheter apparatus can distort and distend the tissues at an around the site of the procedure. The clinician will usually plan the procedure with an angiogram of the site, with vessel lumens and the site requiring repair distinctly identified by the presence of a contrast medium. Such advance mapping of the procedure is of minimal assistance when the anatomy is distorted during the procedure to different dimensions and locations than appeared in the preparatory angiogram. As a result, the stent may ultimately be deployed in a slightly different location or orientation than that which was intended. This can be particularly problematic when the implant site is at or near the confluence of several blood vessels.

An example of such a problematic deployment is illustrated in FIG. 1. In this case it was intended to deploy the stent 150 at the terminus of the left common iliac vein 12, just below its jointure with the contralateral common iliac vein 11 and the inferior vena cava 10. However, as the drawing illustrates, the stent was mispositioned such that it projects into the inferior vena cava 10 and also crosses the flow pathway of the right common iliac vein 11, where it can impede the venous flow and possibly cause right iliac vein thrombosis. Stents that extend into other flow pathways, termed “jailing” by interventional radiologists and vascular surgeons, can impede flow of the crossed pathway. Bare metal stents will allow blood to traverse the stent interstices which in time can become occluded by fibrin buildup, further increasing the resistance to flow. Eventually the flow pathway will be fully impeded and the jailed vessel will occlude. If this occurs in the iliac veins' confluence it will result in contralateral deep vein thrombosis.

Stents will also exhibit effects of aging in the body. Stent fracture is a recognized complication of intravascular stent implantation, with the primary cause being mechanical stress where repetitive contractions expose stents to forces such as compression, torsion, kinking, elongation, bending, and shear stress. These forces can lead to mechanical fatigue and eventual fracture of the stent material. Stent fractures can lead to a range of clinical complications, depending on their severity and location.

Fractured stents can trigger thrombosis due to abnormal endothelialization and local mechanical irritation. This can result in occlusion of the stent which, in the case where the stent crosses into the contralateral flow pathway, causes stenosis or occlusion of flow. Ultimately this problem can result in contralateral venous thrombosis and, if in the pelvis, lower extremity deep vein thrombosis.

### SUMMARY OF THE INVENTION

In one aspect of the disclosed concept, a stent deployment apparatus is configured to be contained within a deployment sheath, and deploy a vascular stent at a deployment site in a blood vessel of vasculature of a body. The stent deployment apparatus includes a delivery sheath, a pusher shaft coupled to the delivery sheath, and at least one stent assist bar associated with the delivery sheath and movable between a retracted position and a deployed position responsive to separation from the deployment sheath, the at least one stent assist bar being adapted to be positioned in relation to a known feature of the vasculature. The pusher shaft is movable with respect to delivery sheath in order to vary the spatial relationship between the vascular stent and the at least one stent assist bar, and thereby dispose the vascular stent to a desired spacing within the blood vessel.

In another aspect of the disclosed concept, a stent deployment apparatus for deploying a vascular stent at a deployment site in a blood vessel of vasculature of a body is provided. The stent deployment apparatus comprises the vascular stent, a delivery catheter, and an intravascular balloon located a desired distance proximal to a proximal end of the vascular stent when partially deployed from the delivery catheter. The intravascular balloon is adapted to engage an ostium of the blood vessel when inflated and position the vascular stent at a desired location within the blood vessel.

### BRIEF DESCRIPTION OF THE DRAWINGS

FIG. 1 is an illustration of an inaccurately deployed stent obstructing the flow at a confluence of blood vessels.

FIG. 2 illustrates an angiogram of the common iliac veins and their confluence with the inferior vena cava.

FIG. 3 illustrates a stent with an attached stent assist bar in accordance with the present invention.

FIG. 4 illustrates the stent and stent assist bar of FIG. 3 when packaged for catheter deployment.

FIG. 5 illustrates the stent and stent assist bar of FIG. 4 when packaged for delivery in a deployment sheath with a pusher rod.

FIGS. 6 through 11 is a sequence of illustrations of the deployment of the stent with the attached stent assist bar of FIG. 3.

FIGS. 12 through 14 is a sequence of illustrations of the deployment of a stent a precise distance away from the confluence of two blood vessels.

FIG. 15 illustrates a stent packaged for delivery with a partially opened stent assist bar.

FIGS. 16 through 18 illustrate the deployment of the packaged stent and stent assist bar of FIG. 15.

FIG. 19 illustrates the packaging of a stent of the present invention with a vascular wire delivered in a wire guide catheter that serves as the stent assist bar.

FIGS. 20 through 27 illustrate the delivery of a catheter by the stent delivery system of FIG. 19.

FIG. 28 illustrates a stent delivery system of the present invention with a stent assist bar extending from the trailing end of the stent.

FIGS. 29 through 34 are a series of drawings illustrating the deployment of the stent delivery system of FIG. 28 at an orthogonal confluence of blood vessels.

FIGS. 35 and 36 illustrate a variation of the stent delivery system of FIG. 28 in which the stent assist bar is attached to the stent pusher rod and removed from the body with the withdrawal of the catheter delivery system.

FIGS. 37 to 42 illustrate a variation in configuration of FIG. 19 where a vascular wire is delivered in a wire guide catheter that serves as the stent assist bar configured with an extended tip design.

FIGS. 43 and 44 illustrate the deployment of a stent with an extended tip design in accordance with the principles of the present invention.

FIG. 45A illustrates a stenosis within an artery, FIG. 45B illustrates suboptimal stent placement in the artery, and FIG. 45C illustrates optimal stent placement in the artery.

FIG. 46D illustrates a stenosis within a branch vessel, FIG. 46E illustrates suboptimal stent placement within the branch vessel, and FIG. 46F illustrates optimal stent placement covering the stenosis in the branch vessel.

FIG. 47G illustrates a branching vessel and a stenosis, FIG. 47H illustrates suboptimal stent placement covering entry to the vessel, and FIG. 47I illustrates optimal stent placement covering the stenosis without covering the branching vessel.

FIG. 48J illustrates suboptimal stent placement within a target vessel and not communicating with a graft opening and not communicating with the target vessel, and FIG. 48K illustrates optimal placement of both stents.

FIG. 49 illustrates stent deployment apparatus of the present invention within a deployment sheath and with the stent assist bars folded back in the delivery position.

FIG. 50 illustrates a stent deployment device of the present invention with the undeployed stent abutting an end plate with a pusher shaft inside of the deployment sheath and an adjusting thread piece moving the stent away from the stent adjustment bars.

FIG. 51 illustrates the stent deployment device with the adjusting threads extending further into the sheath and with a smaller distance between the end plate and the stent adjustment bars.

FIG. 52 illustrates the stent deployment device with the adjusting threads furthest into the sheath and with no space between the end plate and the stent adjustment bars.

FIG. 53 illustrates the stent deployment device deployed within a vessel with the stent assist bars abutting a main vessel side wall.

FIG. 54 illustrates a stent in its expanded state and positioned a distance from the main vessel wall.

FIG. 55 illustrates the final stent placement positioned a distance from the vessel wall and with the deployment device removed.

FIG. 56 illustrates a second embodiment of the present invention where there is a second adjustment mechanism with threads on the outer surface of the deployment sheath for adjusting the stent assist bars.

FIG. 57 illustrates the embodiment of FIG. 56 where the stent assist bars are advanced by means of the threads to create a desired distance from the stent assist bars to the end plate and proximal stent.

FIG. 58 illustrates further advancement of the threaded device with greater distance between the stent assist bars and the end plate and proximal stent.

FIG. 59 illustrates the deployment device positioned within a vessel with the stent assist bars opposing the wall of the main vessel and the proximal stent flush with the ostium of the branching vessel.

FIG. 60 shows the stent of FIG. 59 in its expanded state.

FIG. 61 illustrates a stent positioned at the opening of a branching vessel flush with the main vessel wall.

FIG. 62 illustrates an alternative position of the stent with the threaded piece fully extended, creating a desired distance from the wall of the main vessel to the outward projecting stent.

FIG. 63 illustrates the stent of FIG. 62 in its expanded state.

FIG. 64 illustrates an expanded stent projecting a distance from the outer portion of the main vessel wall into the main vessel lumen.

FIG. 65 illustrates an implementation of the present invention where the delivery catheter comprises a catheter with a balloon and a stent.

FIG. 66 illustrates the balloon of FIG. 65 partially inflated.

FIG. 67 illustrates the deployment device of FIG. 66 with the balloon fully inflated.

FIG. 68 illustrates the deployment device of FIG. 67 entering a vessel with the balloon deflated.

FIG. 69 illustrates the deployment device of FIG. 68 entering a vessel with the balloon partially expanded.

FIG. 70 illustrates the deployment device of FIG. 69 with the balloon) fully expanded and abutting the wall of the main vessel, with the stent inside of the branching vessel and with the proximal end even with the main vessel wall.

FIG. 71 illustrates the deployment device of FIG. 70 with the stent in its expanded state.

FIG. 72 illustrates the stent of FIG. 71 expanded with the proximal end even with the main vessel wall.

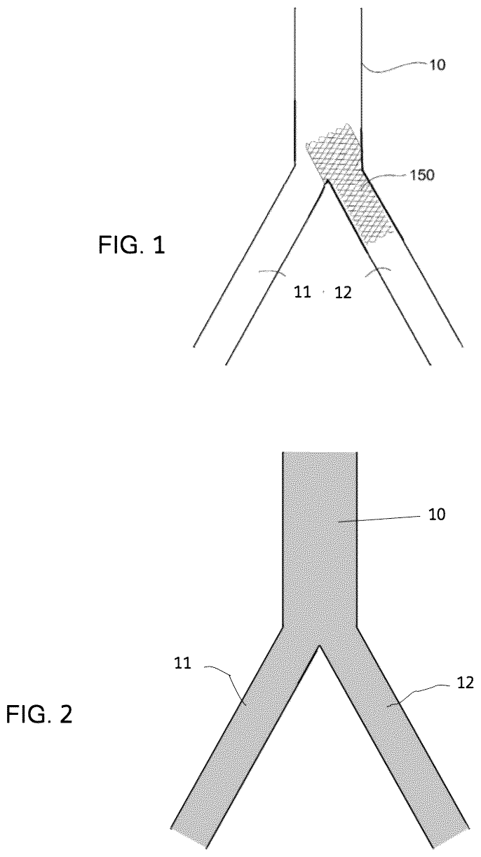

### DESCRIPTION OF THE PREFERRED EMBODIMENTS

As employed herein, the term “coupled” shall mean connected together either directly or via one or more intermediate parts or components.

As employed herein, the term “number” shall mean one or an integer greater than one (i.e., a plurality).

A vascular stent is preferably implanted in accordance with the present invention by an image-guided catheter-borne procedure. Prior to the procedure, an angiogram is acquired by fluoroscopy, using contrast media which distinctly depicts blood flow and flow abnormalities in the vessels of interest. The width of the blood in a lumen will appear narrowed where plaque has built up on a vessel wall, for instance. FIG. 2 depicts an angiogram of the oblique confluence of the right and left common iliac veins 11 and 12 with the inferior vena cava 10. The gray shading in the vessels illustrates how distinctly the contrast-enhanced blood flow of the vessels stands out against the surrounding area. The angiogram is used by a clinician to plan the deployment of a stent, mapping the exact location where a stent should be implanted. During the deployment procedure, a digital form of the angiogram can be overlaid over the real-time fluoroscopic image or displayed alongside the real-time image to help guide the placement of the stent.

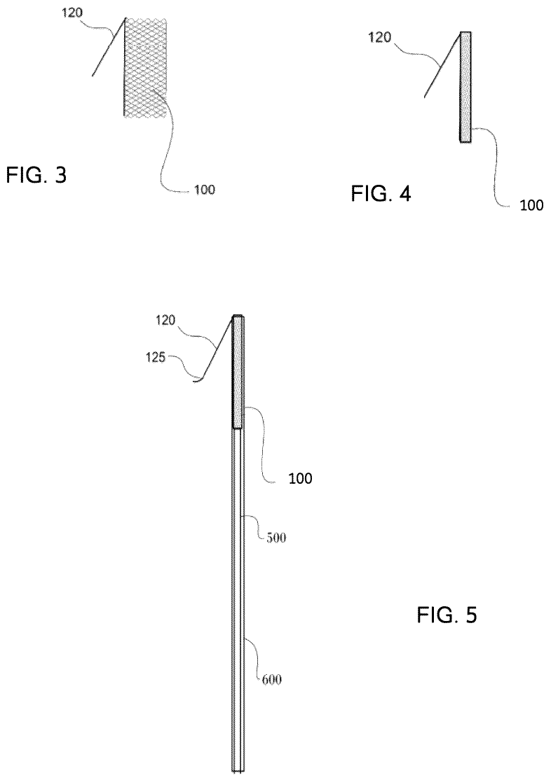

But, as previously mentioned, the forces applied in the body at the site of a procedure by the motion and manipulation of the catheter-borne instrumentation can distort and distend the local anatomy and its vasculature, so that the anatomy at the time of the procedure will not exactly match the anatomical map of the preparatory angiogram. This problem is overcome by an implementation of the present invention such as the stent 100 shown in FIG. 3.

In this illustration the stent is shown in its fully expanded form as it appears when deployed in a blood vessel. In the implementation of FIG. 3 the stent 100 has a stent assist bar 120 attached to a side of the stent. The stent assist bar in this embodiment comprises a vertical section (also called a base section) affixed to the side of the stent (in this embodiment), and an angled section which extends outward from the vertical section and is joined to the base section at an apex. This apex has an angle of less than 180 degrees when in a relaxed and unloaded state. The apex can be loaded by forces to collapse the apex, such as to make the stent assist bar narrower during placement. In one embodiment, a removable sheath provides these forces. When forces are removed, the apex returns to its unloaded angle. This angle can be obtuse, acute or a right angle.

The stent assist bar may be made of, for instance, stainless steel, nickel-titanium (nitinol), cobalt-chrome alloys, tantalum and tungsten which can be coated with materials such as PTFE, polyurethane and other hydrophilic polymer coatings. The stent assist bar may also made of materials the dissolve or reabsorb in the vascular system such as polymers like Poly (L-lactide) (PLLA), Poly (lactide-co-glycolic acid), Poly glycolic acid and Poly-E-caprolactone. Metals such as Magnesium and its alloys, Zinc and its alloy and Iron as well as other materials such as Tyrosine poly carbonate, Salicylic acid2 and Polydiolcitrate: A light-curable material incorporating methacrylate groups. The stent assist bar may be formed as an integral part of the stent during the stent manufacturing process, or it may be formed separately and attached to the stent by spot welding, soldering, or being crimped onto the stent. It may also be adhesively attached to the stent. FIG. 4 shows the stent and stent assist bar with the stent in its fully collapsed form for insertion into a catheter-borne delivery sheath 600 as shown in FIG. 5. The collapsed stent is seen to be located at the upper end of the sheath, with the angled section 120 of the stent assist bar located outside of the sheath. In this implementation the angled section 120 terminates in a curved distal tip 125 which protects the vessel during stent deployment. The stent 100 is mounted on a pusher rod 500 within the sheath 600, which is advanced to eject the stent from the sheath for stent deployment.

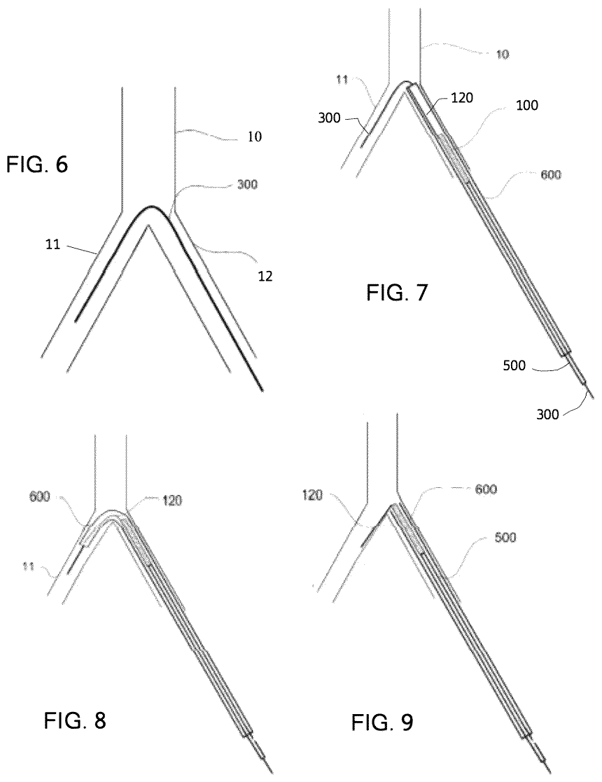

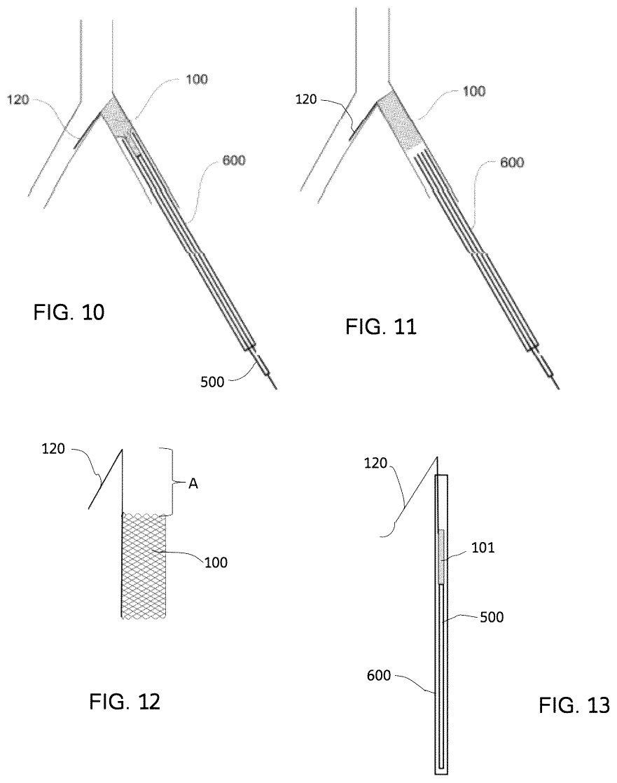

The positioning of the stent within a blood vessel and its deployment at an intended site within the vessel is illustrates in the sequence of drawings of FIGS. 6 through 11. In FIG. 6 a guide wire 300 is first advanced through the common iliac vein 12 and crosses through its confluence with the inferior vena cava 10 and is further advanced into the contralateral common iliac vein 11. FIG. 7 then shows the advancement of the delivery system of FIG. 5 over the guidewire 300. In this particular implementation the stent is not located at the very top of the deployment sheath 600, but in a retracted position with the stent assist bar 120 extending forward of the stent inside the sheath. In FIG. 8 the delivery system has been further advanced until the extended stent assist bar 120 is located in the contralateral common iliac vein 11. The stent is now positioned with its leading edge at the confluence of the iliac veins. The deployment sheath 600 is then pulled back while holding the pusher rod 500 in a stationary position, uncovering the stent assist bar 120 which is in the contralateral vessel. The deployment system is pulled back further, snugging the stent assist bar 120 against the medial wall of the contralateral iliac vein as shown in FIG. 9. With the stent assist bar now firmly positioning the tip of the stent at the confluence of the common iliac veins, the deployment sheath is pulled back further while maintaining the position of the pusher rod 500 which begins to eject the stent from the deployment sheath as shown in FIG. 10. The self-expanding stent 100 begins to expand as shown in FIG. 10 and is shown fully deployed in FIG. 11. The deployment system including the guide wire 300, the pusher rod 500, and the deployment sheath 600 is then withdrawn from the body.

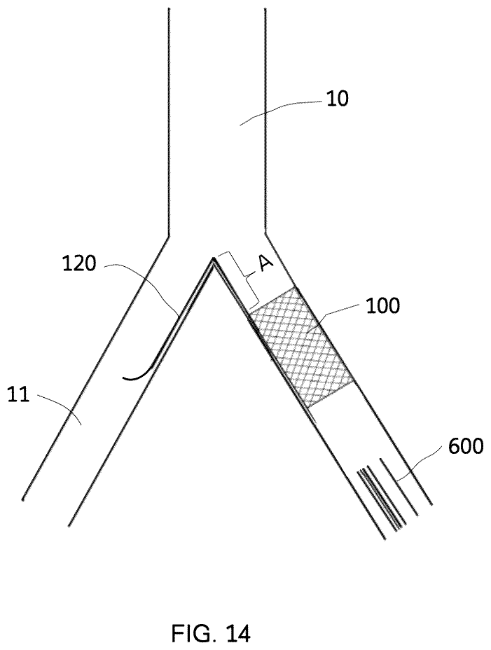

FIGS. 12 through 14 illustrate the structure and deployment of a stent 100 which again has a stent assist bar 120 attached to the side of the stent as in FIG. 3, but in this case the stent assist bar is configured so as to deploy the stent at a precisely determined location below the confluence of blood vessels. In this example the section of the stent assist bar 120 which is attached to the side of the stent extends above the stent 100 for a distance “A” before the angled section extends outwardly. This enables the stent to be positioned with its center located a predetermined distance below a vessel confluence. For example, suppose the stent has a length of 12 mm, and it is desired to deploy the stent with its center over a vessel defect which is located 10 mm below the confluence of the common iliac veins. In that case, half of the stent length is 6 mm and, if dimension A is set to be 4 mm, then the stent will be precisely deployed with its center over the vessel defect. The stent of FIG. 12 may be provided with a straight stent assist bar extending from the stent, which is then creased by the clinician exactly 4 mm above the end of the stent to provide dimension A from which the angled section extends. Alternatively, the stent may be manufactured with the stent assist bar accurately dimensioned as shown in FIG. 12 for the procedure. FIG. 13 shows the collapsed stent 101 and its pusher rod 500 positioned in the delivery sheath 600 and the stent assist bar 120 extending out the end of and away from the sheath. When the stent is advanced so that the stent assist bar 120 with its angled bend is positioned at the confluence of the common iliac veins as shown in FIG. 14, it is seen that the center of the deployed 12 mm stent 100 is precisely positioned 10 mm below the confluence of the common iliac veins (A=4 mm plus half of the stent length=6 mm). The deployment sheath 600 and the rest of the deployment apparatus is then withdrawn from the site of the deployed stent.

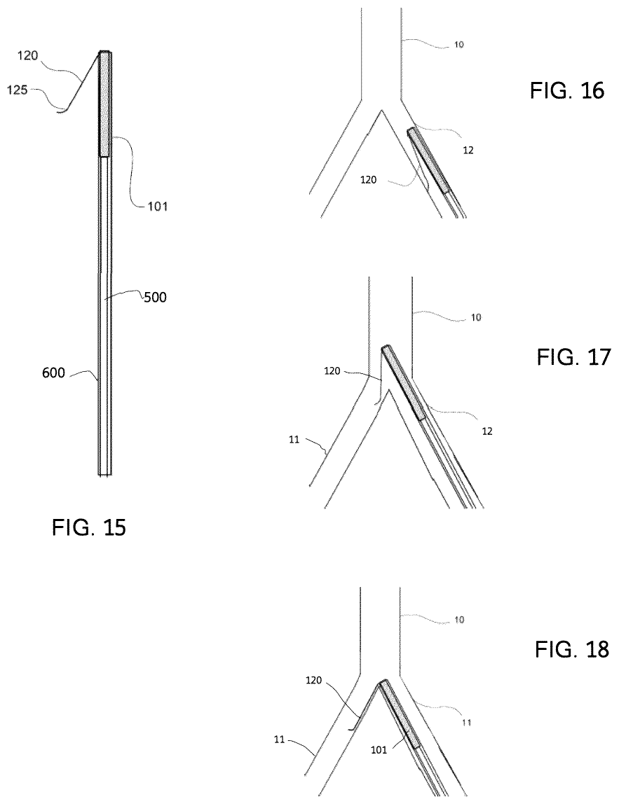

FIGS. 15 through 18 illustrate another implementation of the present invention in which the angled section 120 of the stent assist bar is located outside of the deployment sheath during the entirety of deployment. The stent assist bar 120 may take one of three configurations in this example. It may be attached to the stent as in the implementation of FIG. 3; it may be attached to the deployment sheath 600; or it may comprise a guide wire not attached to the deployment system and extending the full length of the catheter. In the first case the stent assist bar will remain in the body; in the latter cases the stent assist bar will be removed from the body with the withdrawal of the stent deployment system from the body. In FIG. 15 a stent 101 is mounted on pusher rod 500 and enclosed in deployment sheath 600. The angled section of the stent assist bar 120 has a curved distal tip 125 to protect the vessel during deployment. In FIG. 16 the delivery system is seen advancing inside of the common iliac vein 12 and the stent assist bar 120 is continuously open and engaging the medial wall of the vein 12. In FIG. 17 the system is shown further advanced into the inferior vena cava 10 with the stent assist bar 120 extending into the contralateral iliac vein 11. The deployment sheath and pusher rod are then pulled back as shown in FIG. 18, seating the stent assist bar 120 against the medial wall of the iliac vein. The stent is then deployed at the top of the left common iliac vein 11 where it is positioned as shown in FIG. 18.

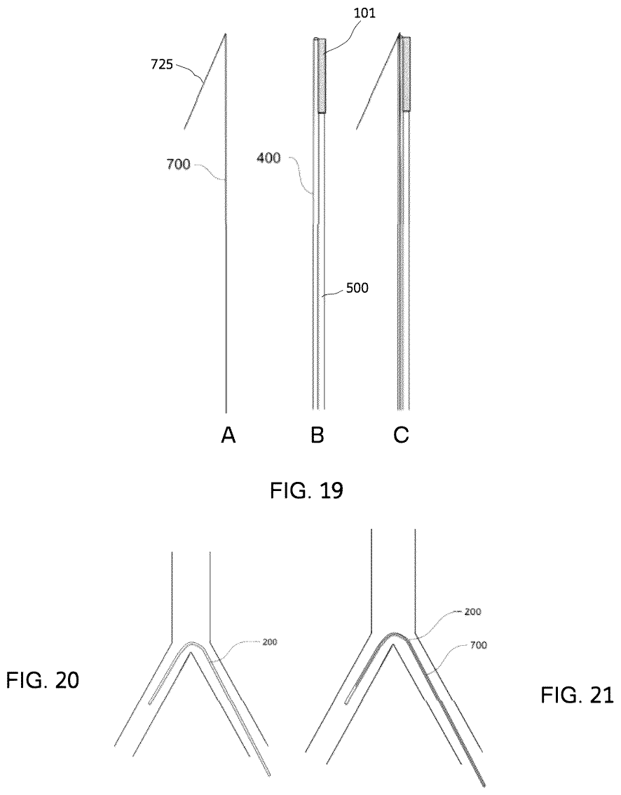

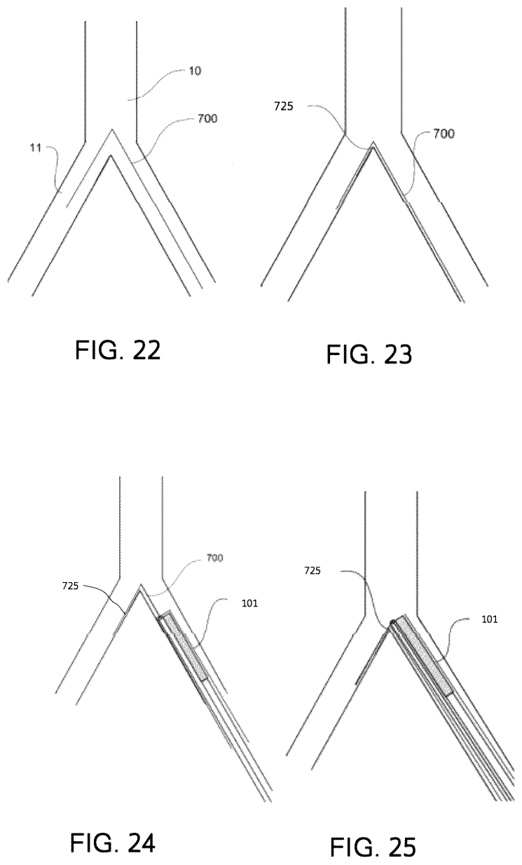

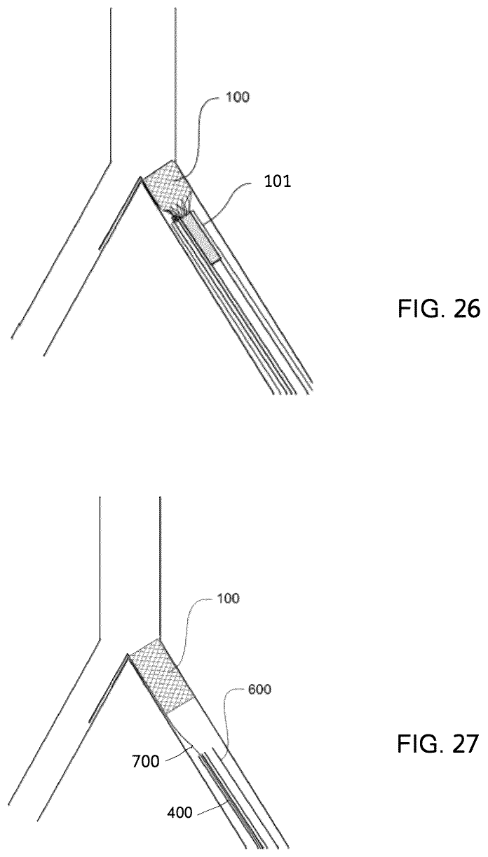

Another embodiment of a stent deployment system of the present invention is shown in FIG. 19 where the stent 101 and pusher rod 500 are combined with a wire guide catheter 400 as shown in FIG. 19B. The wire guide catheter accepts a vascular wire 700 (which can act as a form of the stent assist bar) that has a preformed distal angled section 725 shown in FIG. 19A. The fully assembled deployment system is shown in FIG. 19C. The deployment of this implementation of the present invention is illustrated in FIGS. 20 through 27. A standard vascular catheter 200 is advanced from the site of introduction into the body to the contralateral iliac vein as shown in FIG. 20. The angled wire 700 is then advanced through the catheter 200 and positioned in the iliac vein as shown in FIG. 21 and the catheter 200 is removed, leaving the angled wire 700 as shown in FIG. 22. The angled wire 700 is pulled back until it engages the medial confluence of the iliac veins and the inferior vena cava as shown in FIG. 23. The deployment system including a stent 101 is then advanced over the angled wire 700 as shown in in FIG. 24 and advanced until it abuts the apex of the wire angle positioned at the confluence of the common iliac vessels as shown in FIG. 25. The delivery system is then withdrawn as shown in FIG. 26, causing the stent 100 to partially deploy and then fully deploy as shown in FIGS. 26 and 27. The angled wire 700 is then removed and the rest of the deployment system withdrawn from the vascular system as illustrated in FIG. 27.

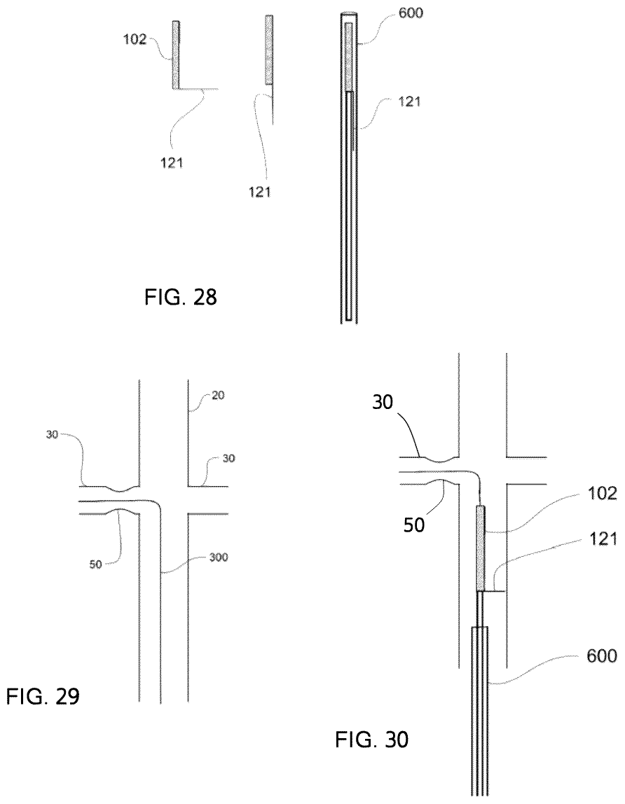

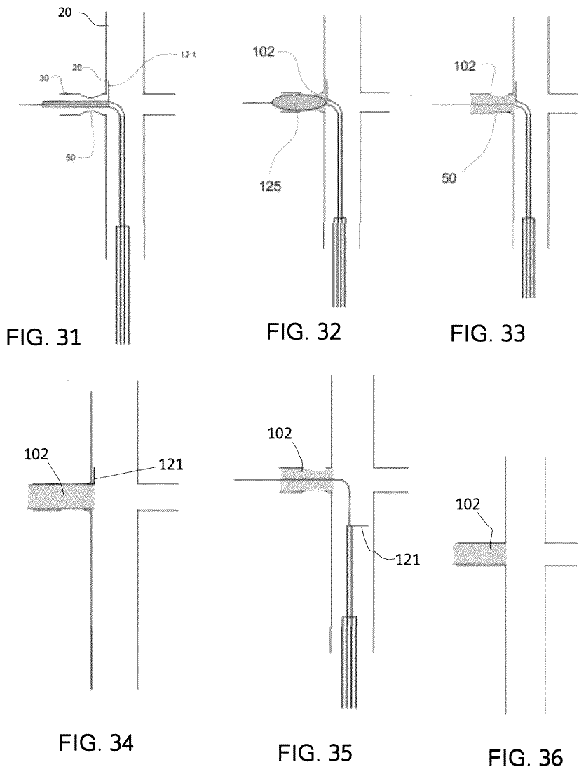

Branching vascular vessels can result in the need for a similarly accurate stent deployment system but with a configuration where the stent assist bar is positioned on the trailing end of the stent. An implementation of the present invention with a backend stent assist bar 121 is shown in FIG. 28 in its extended (left) and non-extended (center) position, as well as within a deployment sheath 600 (right). FIG. 29 illustrates an example of a branching vascular structure consisting of the abdominal aorta 20 and its perpendicularly aligned branching renal arteries 30. In this example there is a stenosis 50 in the left renal artery branch. A guide wire 300 is advanced through the aorta and into this left renal artery branch as shown in FIG. 29. FIG. 30 shows the deployment system of FIG. 28 advanced over the guide wire and pulled back to reveal unexpanded stent 102 and the opened stent assist bar 121 at the end of the pusher rod. The pusher rod 500 is then advanced over the guide wire to position the stent within the narrowing 50 of the artery 30 as shown in FIG. 31. The attainment of this position is indicated to the clinician when the extended stent assist bar 121 contacts the wall of the aorta 20 as illustrated in FIG. 31.

The stent 102 is of the non self-expanding type and a balloon catheter 125 is used to expand the stent to its desired size. The deflated balloon is inserted into the stent and inflated to expand the stent to its desired size, which in this example opens the stenotic region of the renal artery as illustrated in FIG. 32. The balloon is then deflated and withdrawn, leaving the expanded stent 102 with its stent assist bar in place as shown in FIG. 33, followed by removal of the remaining deployment apparatus as illustrated in FIG. 34. As an option, the stent assist bar can be made of resorbable/dissolvable materials, as noted above.

FIGS. 35 and 36 illustrate a variation of this procedure in which the stent assist bar 121 is attached to the pusher rod rather than the stent, and is removed with withdrawal of the deployment system as shown in FIG. 35. This leaves the expanded stent 102 in place without a remaining stent assist bar as shown in FIG. 36.

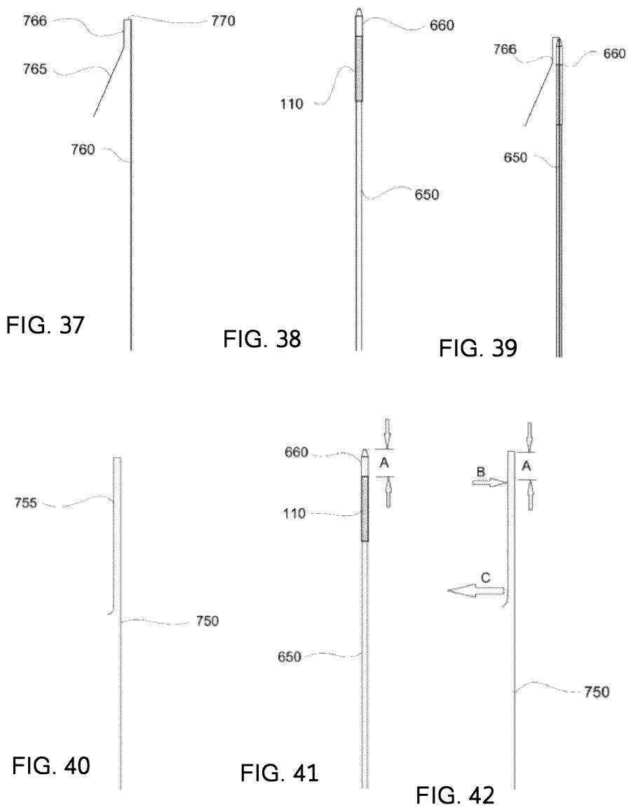

Vascular stents which are currently being marketed have various configurations of the deployment catheter tip, which are designed to retain the unexpanded stent compactly packaged and positioned for catheter deployment and subsequent implantation. The following implementations enable the delivery of these various stent configurations with the guidance of a stent assist bar of the present invention, providing precise placement of different commercially available stent packages. One such stent package design is illustrated in FIG. 38 and comprises an unexpanded stent 110 with an extended tip 660 inside a delivery catheter 650. This extended tip design requires a modification of the stent assist bar to accommodate the extra length of the stent packaging. As shown in FIG. 37 a wire 760 has a terminal blunted end 770 with a retrograde extension piece 766 which terminates in an angled stent assist bar 765. The fully assembled stent, catheter and stent assist bar is shown in FIG. 39 as it appears when ready for stent deployment.

Rather than require a clinic to stock a variety of differently shaped stent assist bar wires 760 for a variety of different stent designs, a preferred implementation starts with a common wire 750 with a bendable section 755 as shown in FIG. 40. When the clinician sees the design of the stent packaging for a given procedure, the bendable section 755 is then formed to match the dimensions of the stent packaging. FIG. 41 shows the extended tip design 660 of the stent packaging being measured and found to have a length A. An arrow B in FIG. 42 shows the point at which the bendable section is bent outward in the direction indicated by arrow C to accommodate the length A of the extended tip. The now-customized wire 750 is thus formed and dimensioned to provide stent assist bar guidance for the delivery of the stent packaging of FIG. 41. The bent configuration enables engagement into a contralateral vessel during delivery and the unbent section of length A will accommodate the extra tip length of the stent packaging. With the system in position the distal end of the stent will approximate the edge of the patient's vessel for accurate deployment.

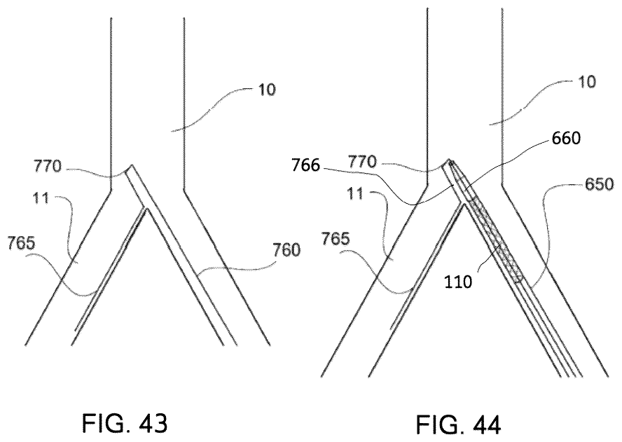

When treating a patient with the wire 760 shown in FIG. 37 or the customized wire 750 shown in FIG. 42, the fully assembled stent deployment system can be advanced to the site of the procedure, but preferably the wire 750 or 760 is advanced into the vessel first and snugged down against the medial vessel wall as shown in FIG. 43. The stent delivery catheter 650 is advanced over the wire 750 or 760 until the distal tip 660 abuts the blunted terminal end 770 of the wire as shown in FIG. 44. As this drawing clearly shows, the distal end 660 of the stent then matches the angle of the extension piece 766, with the stent assist bar 765 positioned in the contralateral vessel 11. The end of the stent 110 is position directly adjacent to the edge of the vessel for accurate deployment. The stent can then be released as previously described, and the other components of the deployment system withdrawn from the body.

Other variations of the stent deployment system of the present invention will readily occur to those skilled in the art. The stent assist bar can be attached to various components of the deployment system, for instance, including the stent delivery sheath, the stent pusher rod as shown in FIG. 35, or to other components of the deployment system.

In another example, it may be necessary to deploy a stent slightly in or partially outside of the ostium of a vessel. The need to deploy a stent an accurately measured distance from the ostium of a vessel bifurcation would occur if the stenosis was slightly inside the opening. The need to deploy a stent with the end a measured distance outside of the ostium, projecting into the main vessel but partially within a branch vessel, could occur if the stenosis is at or surrounding the bifurcation. Another common procedure needing accurate stent placement is endovascular stent grafts extending from bifurcated limbs. These stents are covered with material and are used to treat thoracic and abdominal aortic aneurysms. The covered sections have exit holes so the stent can be placed with flow through the stent graft to a vessel. These vessels can include the carotids or left subclavian arteries arising from thoracic grafts and mesenteric and renal vessels covered by abdominal aortic stent grafts. Placing these branching bifurcated stents into a vessel requires near-exact positioning where the stent needs to be several millimeters within the stent graft to secure it and the rest extending into the target vessel. In such a procedure the clinician could confirm exact stent coverage when the tolerances are tight with very minimal fluoroscopic visualization.

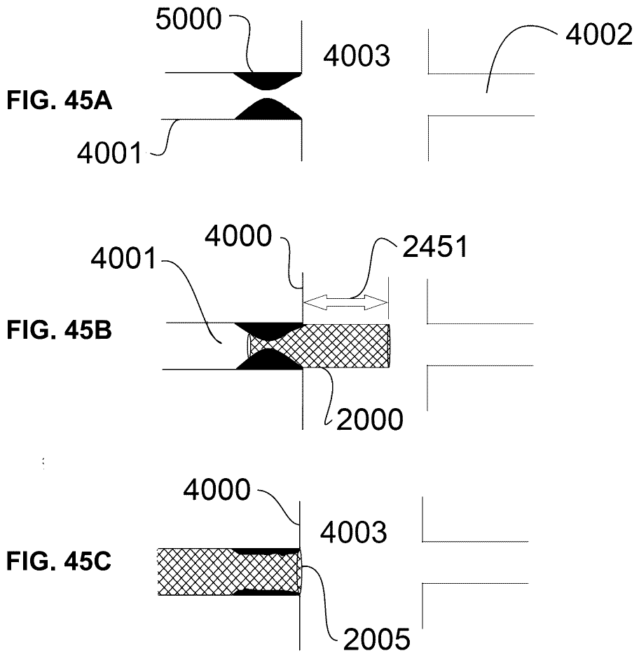

An example of another problematic stent deployment is illustrated in FIG. 45A. In this example, a stenosis 5000 is within the proximal portion of an artery 4001 which intersects a second vessel 4003 and continues from the opposite side of vessel 4003 as indicated at 4002. As this drawing illustrates, the stenosis is immediately adjacent the ostium of vessel 4003. FIG. 45B illustrates suboptimal placement of a stent 2000 in vessel 4001 with the stent projecting into the main lumen of vessel 4003 for a distance 2451. This stent position can create resistance to flow in the main vessel 4003, and could also cause the stent to be pulled out of the branch vessel 4001 due to the forces of flow. FIG. 45C illustrates optimal stent positioning with the stent end 2005 flush with the wall 4000 of vessel 4003.

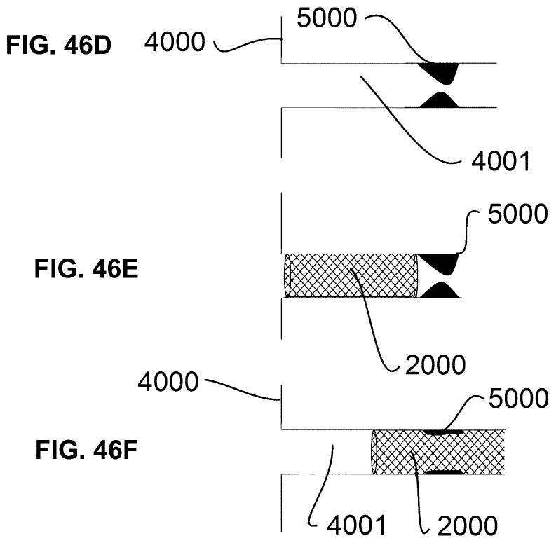

A second type of improper positioning occurs when a stenosis 5000 is located further into the branching vessel 4001 as shown in FIG. 46D. FIG. 46E illustrates suboptimal placement of stent 2000 with the stenosis 5000 not covered by the stent. FIG. 46F depicts optimal placement of stent 2000 covering stenosis 5000.

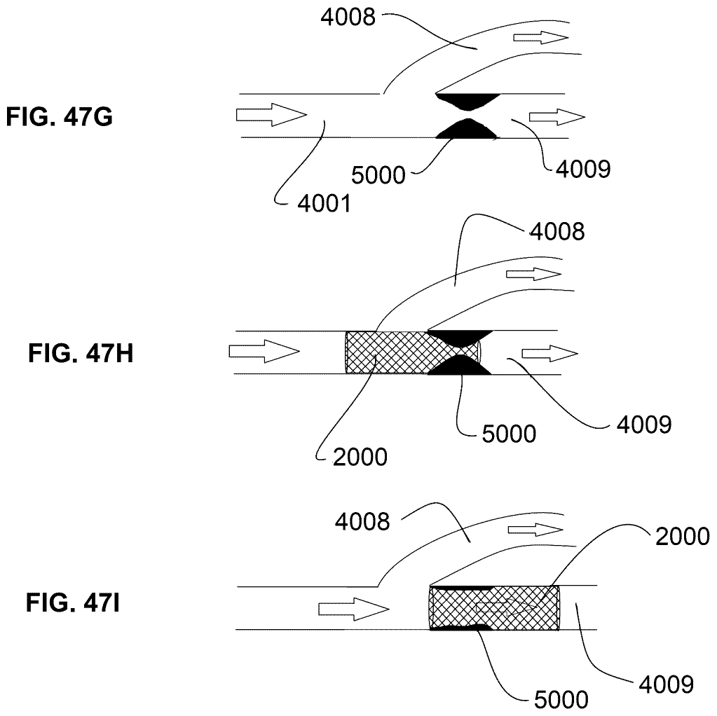

FIG. 47G illustrates a branching vessel 4008 extending from a main vessel 4001 with a stenosis 5000 in the proximal aspect 4009 of the main vessel. Suboptimal placement of stent 2000 is shown in FIG. 47H where the mid and proximal aspects of the stent cover the entry to the branch vessel 4008 which can cause decreased perfusion or vessel occlusion. In comparison, FIG. 47I illustrates optimal placement of stent 2000 covering stenosis 5000 without occluding branch vessel 4008.

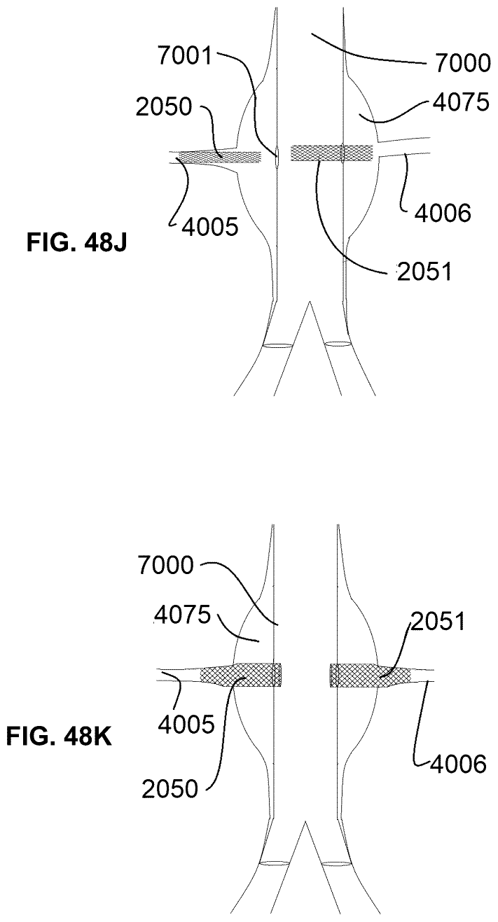

Another very difficult location to accurately place stents, and with inaccurate placement having significant consequences, is within bifurcated stent grafts as shown in FIG. 48. Stent grafts 7000 are conduits that are used to bridge across vascular aneurysms 4075 and/or other vascular pathologies such as aortic dissections. The conduits 7000 contain the flow, so the aneurysm is no longer subjected to high blood pressure, thereby limiting the risk of further aneurysm expansion and eventual rupture. The stent grafts may have side holes 7001 as shown in FIG. 48J where stents can be placed that provide blood flow to vessels arising from within the pathology covered by stent grafts 7000. The stent grafts are typically deployed first, making sure that the side holes 7001 align with the branching vessels 4005 and 4006. Assisted by fluoroscopic imaging, the branching stents 2050 and 2051 are deployed through side holes 7001.

FIG. 48J shows suboptimal placement of stent 2050 within the target vessel 4005 where the deployed stent 2050 does not communicate with graft opening 7001, thereby allowing the stent graft 7000 to leak into the aneurysm 4075. Another type of suboptimal placement of a stent 2051 is shown where the stent does not extend into the target branching vessel 4006, again causing possible leakage into the aneurysm 4075. FIG. 48K illustrates optimal stent placement, where both stents 2050 and 2051 extend slightly into the lumen of the stent graft 7000 and also extend into the target branch vessels 4005 and 4006.

Accordingly it is an object of the present invention to enable precise catheter deployment of stents within blood vessels, preferably to millimeter accuracies. It is a further object of the present invention to provide such accuracy when a stent is to be implanted at or near the location of a confluence of blood vessels in the body.

In accordance with the principals of the present invention, the catheter-borne deployment apparatus for a vascular stent comprises a device which assures that a stent is deployed with precision in relation to a known feature of the vasculature. In a first implementation, the device comprises at least one (i.e., one or a plurality) to guide accurate stent placement. In a second implementation the device comprises a catheter balloon to provide accurate stent placement. In the first implementation the distance between the stent assist bars and a stent which is to be deployed is set by a threaded mechanism. In one configuration the threaded mechanism enables the position of the stent to be adjusted in relation of the stent assist bars. In another configuration the threaded mechanism enables the position of the stent assist bars to be adjusted in relation to the position of the stent. The threaded mechanism can be adjusted prior to catheter insertion or after, so that the stent will be deployed precisely at the stenosis and in desired relation to a vessel bifurcation.

In a preferred implementation stent deployment apparatus for deploying a vascular stent at a deployment site in a blood vessel of vasculature of a body comprises a vascular stent; a delivery sheath; a body (e.g., pusher shaft, threaded adjustment mechanism); stent assist bars, located in relation to the stent, and deployable outward from the deployment apparatus, the stent assist bars adapted to be positioned in relation to a known feature of the vasculature; and optionally a position adjustment mechanism, coupled to the stent deployment apparatus, and adapted to vary the spatial relationship between the vascular stent and the stent assist bars. In an implementation using an intravascular balloon in place of the stent assist bars, the stent deployment apparatus comprises a vascular stent; a delivery catheter; and an intravascular balloon located a desired distance proximal to a proximal end of the vascular stent when partially deployed from the delivery catheter, wherein the intravascular balloon is adapted to engage the ostium of a blood vessel when inflated and position the vascular stent at a desired location within the blood vessel.

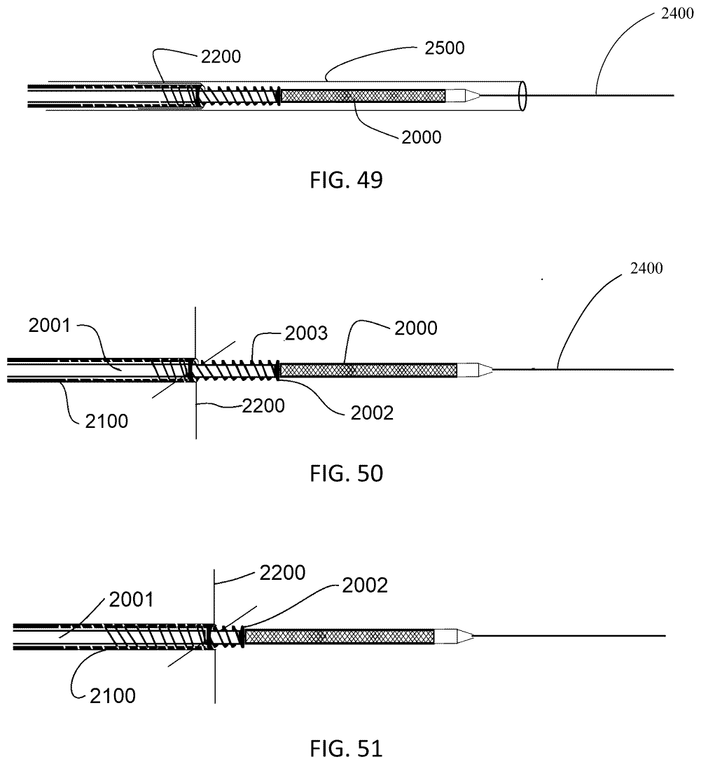

Referring to FIG. 49, an adjustable stent deployment apparatus of the present invention is shown in its stowed condition prior to deployment. Contained within a deployment sheath 2500 are the elements needed to accurately deploy a stent 2000 at a precise location in a blood vessel. The stent 2000 is located at the end of a threaded mechanism and ahead of at least one stent assist bar 2200, which in FIG. 49 are folded back inside the deployment sheath 2500. It will be appreciated that the stent assist bars 2200 are configured to move from a retracted position (FIG. 49) to a deployed position (FIG. 50) responsive to separation and/or removal from the deployment sheath 2500. The stent assist bars 2200 may be made of, for instance, stainless steel, nickel-titanium (nitinol), cobalt-chrome alloys, tantalum and tungsten which can be coated with materials such as PTFE, polyurethane and other hydrophilic polymer coatings.

FIG. 50 shows the apparatus, which comprises the pusher shaft 2001, the delivery sheath 2100, and the stent assist bars 2200, after the deployment sheath 2100 has been withdrawn, with the stent assist bars 2200 now deployed radially. The apparatus is mounted on a central guide wire 2400. Behind the stent 2000 is a pusher shaft 2001 used to deploy the stent. That is, the pusher shaft 2001 is movable with respect to delivery sheath 2100 in order to vary the spatial relationship between the vascular stent 2000 and the stent assist bars 2200, and thereby locate the vascular stent 2000 to a desired spacing within a blood vessel.

The pusher shaft 2001 is surrounded by a threaded piece 2003 which extends from an abutting end plate 2002 at the end of the stent, around the pusher shaft 2001, and into mating thread grooves around the inside of a delivery sheath 2100 (e.g., the pusher shaft 2001 and the delivery sheath 2100 are threadably coupled to each other such that the pusher shaft 2001 is configured to move longitudinally (e.g., without limitation, in and out), with respect to the delivery sheath 2100). Additionally, the stent assist bars 2200 are associated with (e.g., coupled to in the example of FIGS. 49-52) with the delivery sheath 2100, optionally coupled to an end of the delivery sheath 2100.

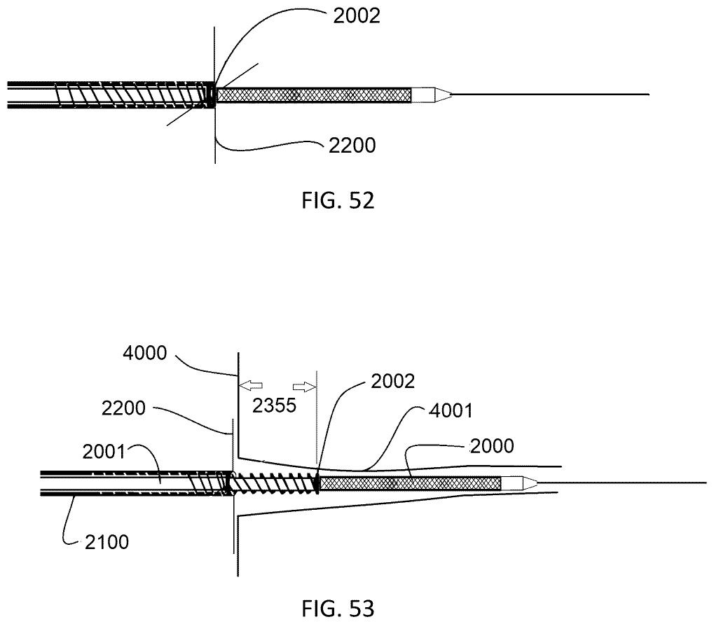

When the pusher shaft 2001 is rotated in one direction the pusher shaft 2001 and stent 2000 are extended from the delivery sheath 2100 and away from the stent assist bars 2200 by a given distance with each rotation, e.g., one millimeter, as illustrated in FIG. 50. When the pusher shaft 2001 is rotated in the opposite direction, the threaded pusher shaft 2003 is withdrawn back into the delivery sheath 2100 and the stent 2000 is moved toward the stent assist bars 2200 as illustrated in FIG. 51. FIG. 52 illustrates the assembly when the threaded pusher shaft 2001 has been threaded fully into the delivery sheath 2100 with the end plate 2002 abutting against the end of the delivery sheath and the end of the stent 2000 in alignment with the stent assist bars 2200. In other words, the end plate 2002 is coupled to an end of the pusher shaft 2001, and when the pusher shaft 2001 is in an enclosed position with respect to the delivery sheath 2100, the end plate 2002 abuts the delivery sheath 2100.

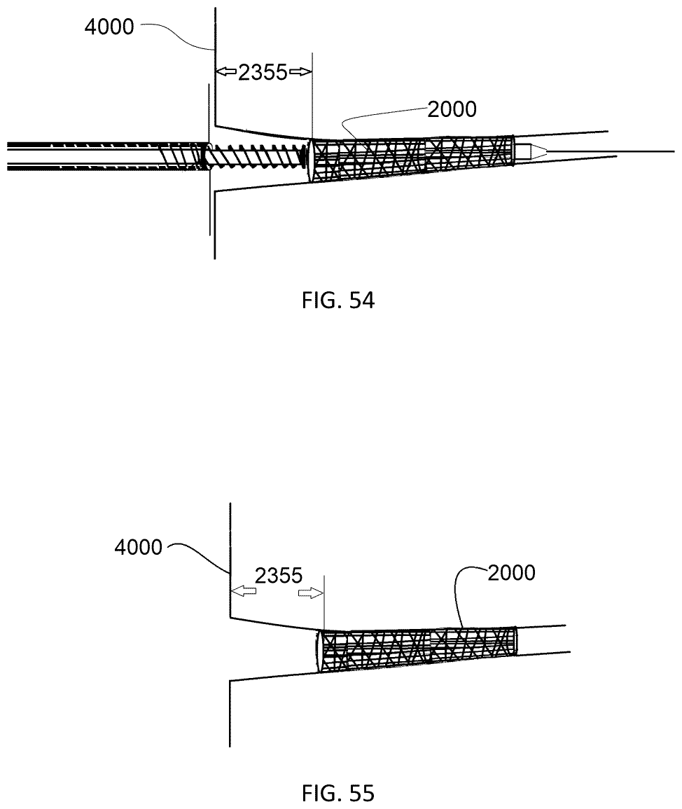

FIG. 53 illustrates the deployment of the apparatus of FIGS. 49-52 in a blood vessel 4001 which bifurcates from the wall 4000 of a main vessel. The stent 2000 is to be positioned within vessel 4001 with its proximal end a precise distance 2355 into the vessel from the bifurcation, as illustrated in FIG. 53. This is achieved by rotating the pusher shaft 2001 until the end plate 2002 is the desired distance from the end of the delivery sheath 2100 from which the stent assist bars 2200 are deployed. The apparatus is inserted into the vessel 4001 until the stent assist bars 2200 abut against the wall 4000 of the main vessel, which results in the stent being positioned at the desired deployment location. The stent 2000 is then deployed as illustrated in FIG. 54 as by means of a balloon, causing the stent 2000 to achieve its expanded state and fixed in position in the blood vessel 4001. FIG. 55 illustrates the final stent placement after the delivery system and catheter have been removed.

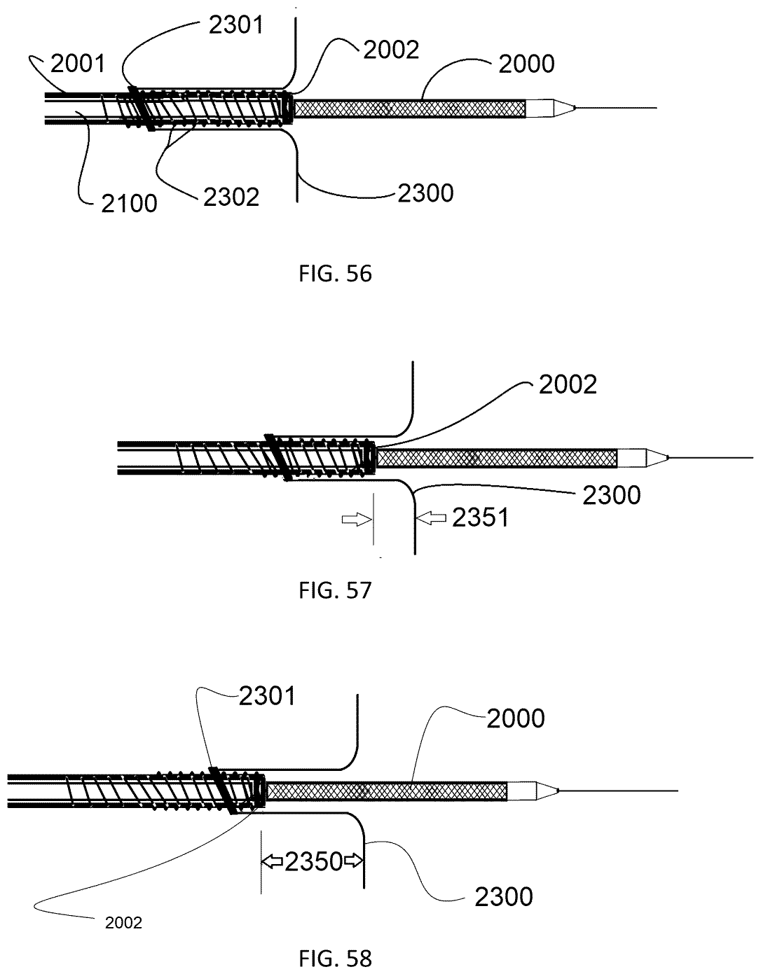

FIG. 56 illustrates a second implementation of a stent delivery apparatus of the present invention in which stent positioning is achieved by adjusting the position of the stent assist bars 2300 relative to the stent 2000. In the implementation of FIG. 56 the stent assist bars 2300 are mounted on and extend from a threaded adjustment mechanism 2301 (e.g., they are associated with the delivery sheath 2100 via being coupled to the threaded adjustment mechanism 2301), and the pusher shaft 2001 (not shown in FIG. 56) is retracted inside the delivery sheath 2100. The adjustment mechanism 2301 is threadably mounted on threads 2302 around the outside of the delivery sheath 2100. As the delivery sheath 2100 is rotated, the adjustment mechanism 2301, and with it the stent assist bars 2300 are advanced or retracted with respect to the delivery sheath 2100 a known distance along the delivery system, e.g., 2 millimeters per revolution.

FIG. 56 illustrates the apparatus when the end plate 2002 at the end of stent 2000 is in alignment with the extended stent assist bars 2300. In FIG. 57 the delivery sheath 2100 has been rotated to position the stent assist bars a precise distance 2351 to the right of the end plate 2002 and the proximal end of the stent 2000. In FIG. 58 the delivery sheath 2100 has been rotated to extend the stent assist bars 230 even further with respect to the end of the stent 2000 so that the proximal end of the stent is a distance 2350 to the left of the stent assist bars 2300.

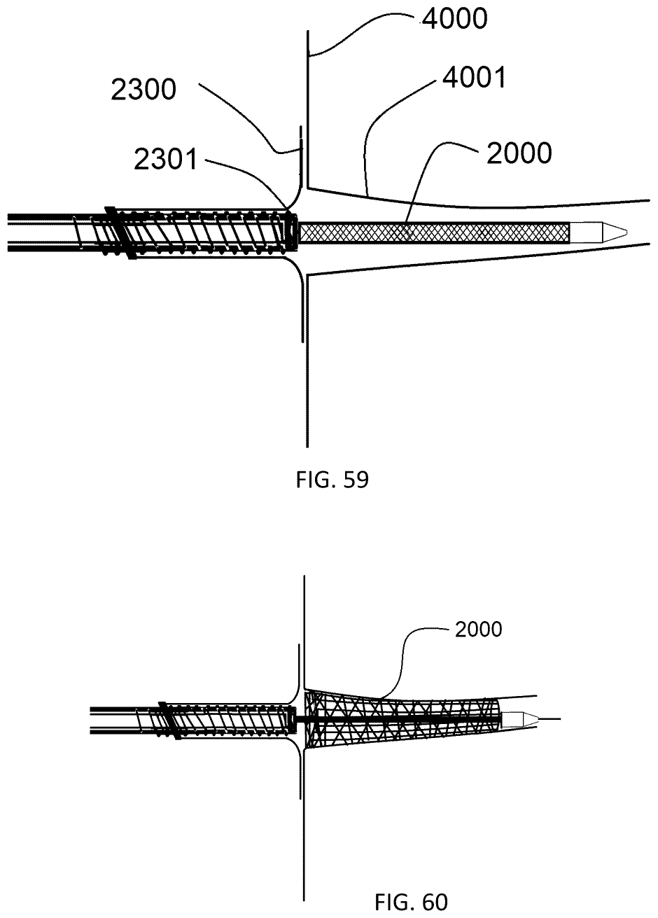

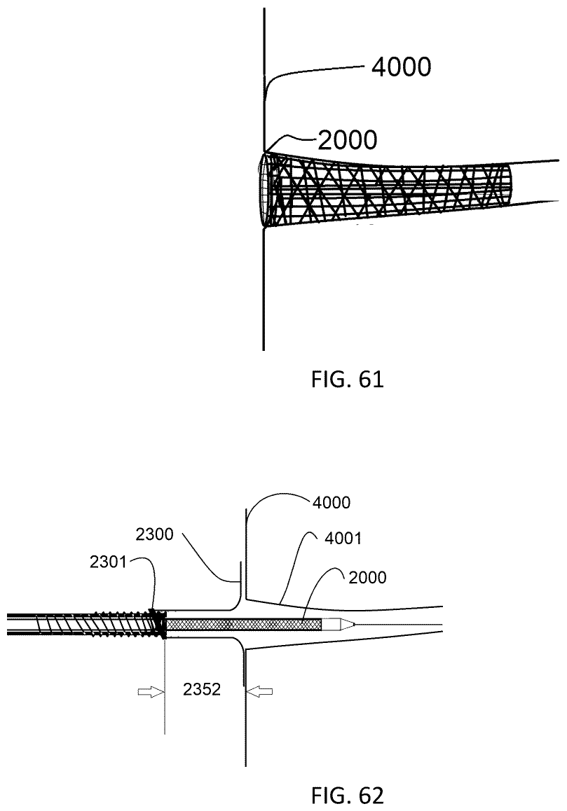

FIGS. 59-64 illustrate two deployment sequences of this implementation. In FIG. 59 the stent 2000 has been adjusted to the position of FIG. 56, with the end of the stent 2000 in alignment with the stent assist bars 2300. The stent 2000 is inserted into bifurcating vessel 4001 with the stent assist bars 2300 contacting the wall 4000 of the main vessel of the bifurcation. This causes the end of the stent 2000 to be aligned with the end of vessel 4001 where it extends from the main vessel. The stent 2000 is now expanded as illustrated in FIG. 60 and the delivery system withdrawn as shown in FIG. 61, leaving the stent 2000 precisely located with its end at the ostium at the main vessel wall 4000.

FIG. 62 illustrates another stent delivery with the adjustment mechanism 2301 for the stent assist bars positioned at the end of the threaded delivery sheath and against the end plate 2002. This positions the end of the stent 2000 a distance 2352 to the left of the extended stent assist bars 2300. When the delivery system is placed with the stent assist bars 2300 in contact with the wall 4000 of the main vessel from which vessel 4001 bifurcates, a portion of the stent 2000 is located in the vessel 4001 and a length 2352 of the stent 2000 extends out of vessel 4001 and into the main vessel. It will be appreciated that a stent delivery of this type is effective for treating vascular aneurysms as illustrated in FIG. 48K.

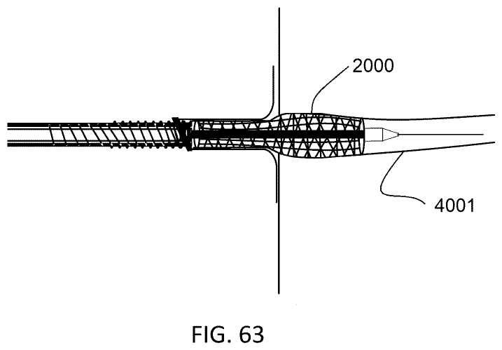

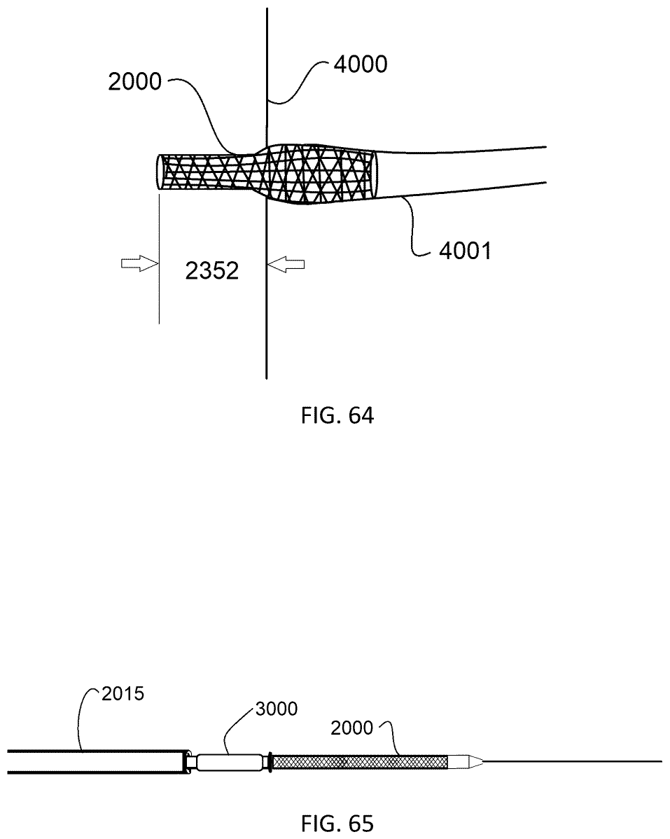

FIG. 63 illustrates completion of the stent delivery of FIG. 62, with the portion of the stent 2000 located within vessel 4001 expanded to secure the stent 2000 in its desired location. FIG. 64 illustrates the final placement of stent 2000 with its expanded portion within vessel 4001 and its length 2352 located within the main vessel to the left of vessel wall 4000.

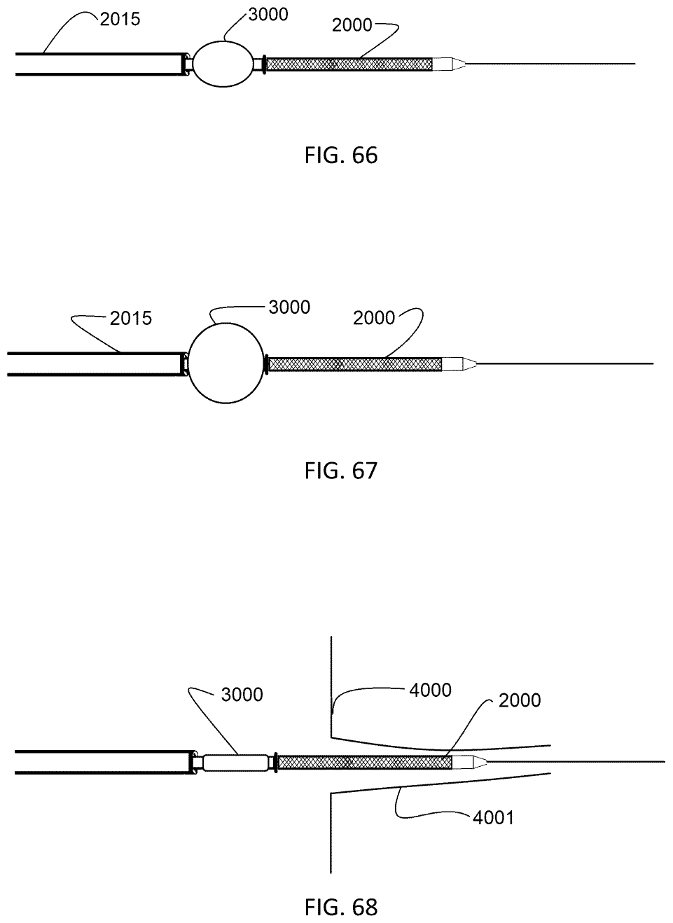

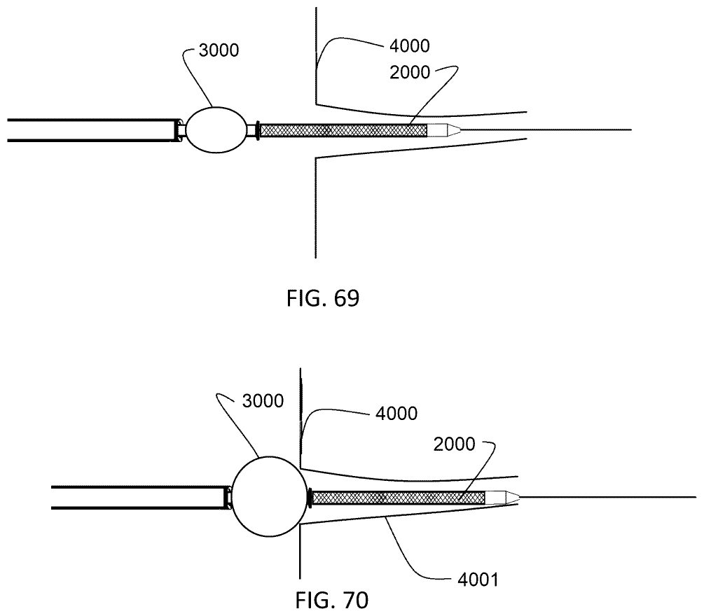

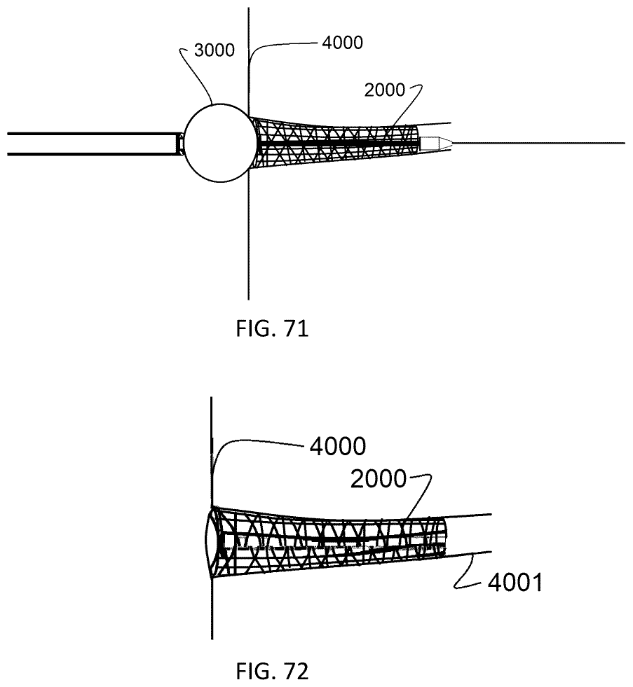

FIGS. 65-72 illustrate an implementation of a stent delivery system of the present invention in which an inflatable balloon 3000 is used to demarcate the opening of a bifurcation from which a stent 2000 is to be accurately deployed. Intravascular balloons are most commonly made from advanced thermoplastic polymers including nylon (polyamide), PEBAX (polyether block amide), polyethylene terephthalate, polyurethane and polyethylene. In FIG. 65, a delivery catheter 2015 delivers an uninflated balloon 3000 and a distal stent 2000 to the surgical site. FIG. 65 illustrates the balloon and stent in their partially deployed configuration, extending forward of the delivery catheter. FIG. 66 illustrates the balloon 3000 partially inflated and FIG. 67 shows the balloon fully inflated. Insertion of the stent deployment system with balloon 3000 is illustrated in FIG. 68, where the stent 2000 is shown entering a branching vessel 4001 from a main vessel 4000. FIG. 69 illustrates the deployment system with the balloon partially inflated, and FIG. 70 shows the balloon 3000 fully inflated and the deployment system advanced until the balloon contacts the branching vessel opening in the main vessel wall 4000. FIG. 71 illustrates the stent 2000 expanded in the desired location at the ostium of the branching vessel. In FIG. 72 the deployment system has been removed and the stent 2000 is positioned within the ostium of the branching vessel 4001 at the desired location from the main vessel 4000.

While the present disclosure has been described with reference to various implementations, it will be understood that these implementations are illustrative and that the scope of the disclosure is not limited to them. Many variations, modifications, additions, and improvements are possible. More generally, implementations in accordance with the present disclosure have been described in the context of particular implementations. Functionality can be separated or combined in blocks differently in various implementations of the disclosure or described with different terminology. These and other variations, modifications, additions, and improvements can fall within the scope of the disclosure as defined in the claims that follow.

What is claimed is:

1. A stent deployment apparatus configured to be contained within a deployment sheath, and deploy a vascular stent at a deployment site in a blood vessel of vasculature of a body, the stent deployment apparatus comprising:
a delivery sheath;
a pusher shaft coupled to the delivery sheath; and
at least one stent assist bar associated with the delivery sheath and movable between a retracted position and a deployed position responsive to separation from the deployment sheath, the at least one stent assist bar being adapted to be positioned in relation to a known feature of the vasculature,
wherein the pusher shaft is movable with respect to delivery sheath in order to vary the spatial relationship between the vascular stent and the at least one stent assist bar, and thereby dispose the vascular stent to a desired spacing within the blood vessel.

2. The stent deployment apparatus of claim 1, wherein the pusher shaft is configured to move longitudinally with respect to the delivery sheath.

3. The stent deployment apparatus of claim 2, wherein the at least one stent assist bar is a plurality of stent assist bars each configured to be about parallel to the delivery sheath when in the retracted position, and about perpendicular to the delivery sheath when in the deployed position.

4. The stent deployment apparatus of claim 2, wherein the pusher shaft is threadably coupled to the delivery sheath, and is configured to move in and out with respect to the delivery sheath.

5. The stent deployment apparatus of claim 2, wherein the at least one stent assist bar extends from an end of the delivery sheath.

6. The stent deployment apparatus of claim 1, further comprising a threaded adjustment mechanism mounted on threads around an outside of the delivery sheath, and wherein the at least one stent assist bar is coupled to the threaded adjustment mechanism in order to advance and retract with respect to the delivery sheath.

7. The stent deployment apparatus of claim 6, wherein the at least one stent assist bar is a plurality of stent assist bars coupled to the threaded adjustment mechanism.

8. The stent deployment apparatus of claim 6, wherein the at least one stent assist bar comprises a first portion and a second portion, wherein the first portion is coupled to the threaded adjustment mechanism, and wherein the second portion is configured to move with respect to the first portion such that in the retracted position the first and second portions are disposed about parallel to one another, and in the deployed position the second portion is disposed about perpendicular to the first portion.

9. The stent deployment apparatus of claim 1, further comprising an end plate coupled to an end of the pusher shaft, and wherein, when the pusher shaft is in an enclosed position with respect to the delivery sheath, the end plate abuts the delivery sheath.

10. The stent deployment apparatus of claim 1, further comprising the vascular stent, wherein the vascular stent is adapted to be deployed when the at least one stent assist bar is in a desired position in relation to the deployment site in the blood vessel.

11. The stent deployment apparatus of claim 10, wherein the vascular stent is adapted to be deployed when positioned partially within the blood vessel and partially within a bifurcating blood vessel.

12. A stent deployment apparatus for deploying a vascular stent at a deployment site in a blood vessel of vasculature of a body, the stent deployment apparatus comprising:
the vascular stent;
a delivery catheter; and
an intravascular balloon located a desired distance proximal to a proximal end of the vascular stent when partially deployed from the delivery catheter,
wherein the intravascular balloon is adapted to engage an ostium of the blood vessel when inflated and position the vascular stent at a desired location within the blood vessel.

13. The stent deployment apparatus of claim 12, wherein the vascular stent is adapted to be deployed when the intravascular balloon is engaging an opening of the blood vessel when inflated.

14. The stent deployment apparatus of claim 13, wherein the intravascular balloon is further adapted to be deflated for withdrawal from the site of the deployed vascular stent.
